(19) United States
(12) Patent Application Publication
(10) Pub. No.: US 2009/0124732 A1
(43) Pub. Date: May 14, 2009

(54) Rylene Tetracarboxylic Acid Diimides Substituted By Cyclic Amino Groups

(75) Inventors: Martin Konemann, Mannheim (DE); Jianqiang Qu, Mannheim (DE); Klaus Mullen, Koln (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/720,409

(22) PCT Filed: Nov. 26, 2005
(86) PCT No.: PCT/EP05/12661

§ 371 Date: May 29, 2007

(30) Foreign Application Priority Data
Nov. 29, 2004 (DE) 10 2004 057 585.1

(51) Int. Cl. C07D 471/02; C08K 5/34; C08K 5/48; C09D 11/00

(52) U.S. Cl. 524/90; 106/31.47; 546/27; 546/37

(57) ABSTRACT

Rylenetetracarboximides of the general formula I

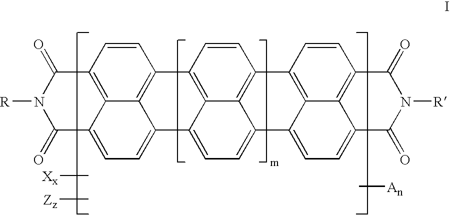

in which the variables are each defined as follows:
R, R′ are each independently hydrogen; optionally substituted C₁-C₃₀-alkyl, C₃-C₈-cycloalkyl, aryl or hetaryl;
R¹ is hydrogen or C₁-C₆-alkyl;
R², R³ are each independently hydrogen; optionally substituted C₁-C₁₈-alkyl, aryl or hetaryl;
A is a 5- to 9-membered ring which is bonded via a nitrogen atom and whose carbon chain may be interrupted by one or more —O—, —S—, —NR¹—, —CO— and/or —SO₂— moieties, to which one or two unsaturated or saturated 4- to 8-membered rings may be fused, whose carbon chain may likewise be interrupted by these moieties and/or —N═, and the overall ring system may be mono- or polysubstituted, and where the A radicals may be the same or different when m>1;
X is chlorine or bromine, and the X radicals may be the same or different when x>1;
Z is optionally substituted C₃-C₂₀-alkyl, C₃-C₂₀-alken-2-yl, C₃-C₂₀-alkyn-2-yl, aryloxy, arylthio, hetaryloxy or hetarylthio, where the Z radicals may be the same or different when z>1;
m is 1 or 2,
n is from 1 to 4 when m=1;
   is from 1 to 6 when m=2;
x is from 0 to 3 when m=1, where n+x+z≦4,
   is from 0 to 5 when m=2, where n+x+z≦6;
z is from 0 to 3 when m=1, where n+x+z≦4,
   is from 0 to 5 when m=2, where n+x+z≦6.

The present invention relates to novel rylenetetracarboximides of the general formula I

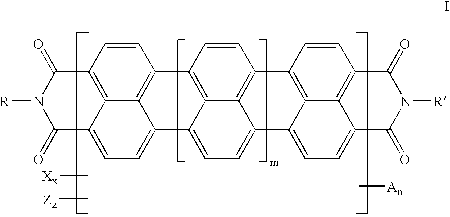

in which the variables are each defined as follows:
R, R′ are each independently:
   hydrogen;
   (1) C₁-₃₀-alkyl whose carbon chain may be interrupted by one or more —O—, —S—, ——NR¹—, —N═CR¹—, —C≡C—, —CR¹═CR¹—, —CO—, —SO— and/or —SO₂— moieties and which may be mono- or polysubstituted by:
   (i) C₁-C₁₂-alkoxy, C₁-C₆-alkylthio, —C≡CR¹, —CR¹≡CR¹₂, hydroxyl, mercapto, halogen, cyano, nitro, —NR²R³, —NR²COR³, —CONR²R³, —SO₂NR²R³, —COOR² and/or —SO₃R²;
   (ii) aryl or hetaryl, to which may be fuzed further saturated or unsaturated 5- to 7-membered rings whose carbon skeleton may be interrupted by one or more —O—, —S—, —NR¹—, —N═CR¹—, —CR¹═CR¹—, —CO—, —SO₂— and/or —SO₂— moieties, where the entire ring system may be mono- or polysubstituted by: C₁-C₁₈-alkyl, C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR², —SO₃R², aryl and/or hetaryl, each of which may be substituted by C₁-C₁₈-alkyl, C₁-C₁₂-alkoxy, hydroxyl, mercapto, halogen, cyano, nitro, —NR²R³, —NR²COR³, —CONR²R³, —SO₂NR²R³, —COOR² and/or —SO₃R²;
   (iii) C₃-C₈-cycloalkyl whose carbon skeleton may be interrupted by one or more —O—, —S—, —NR¹—, —N═CR¹—, —CR¹═CR¹—, —CO—, —SO— and/or —SO₂— moieties and to which may be fuzed further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be mono- or polysubstituted by: C₁-C₁₈-alkyl, C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR², and/or —SO₃R²;
   (iv) a —U-aryl radical which may be mono- or polysubstituted by the above radicals mentioned as substituents for the aryl radicals (ii), where U is an —O—, —S—, —NR¹—, —CO—, —SO— or —SO₂— moiety;
   (2) C₃-C₈-cycloalkyl to which may be fuzed further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be singly or multiply substituted by: the (i), (ii), (iii), (iv) radicals and/or
   (v) C₁-C₃₀-alkyl whose carbon chain may be interrupted by one or more —O—, —S—, —NR¹—, —N═CR¹—, —C≡C—, —CR¹═CR¹—, —CO—, —SO— and/or —SO₂— moieties and which may be mono- or polysubstituted by: C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR² and/or —SO₃R², aryl and/or saturated or unsaturated C₄-C₇-cycloalkyl whose carbon skeleton may be interrupted by one or more —O—, —S—, —NR¹—, —N═CR¹—, —CR¹═CR¹—, —CO—, —SO— and/or —SO₂— moieties, where the aryl and cycloalkyl radicals may each be mono- or polysubstituted by C₁-C₁₈-alkyl and/or the above radicals mentioned as substituents for alkyl;
   (3) aryl or hetaryl to which may be fuzed further saturated or unsaturated 5- to 7-membered rings whose carbon skeleton may be interrupted by one or more —O—, —S—, —NR¹—, —N═CR¹—, —C≡C—, —CR¹═CR¹—, —CO—, —SO— and/or —SO₂— moieties, where the entire ring system may be substituted by the (i), (ii), (iii), (iv), (v) radicals and/or aryl- and/or hetarylazo, each of which may be substituted by C₁-C₁₀-alkyl, C₁-C₆-alkoxy and/or cyano;
R¹ is hydrogen or C₁-C₁₈-alkyl, where the R¹ radicals may be the same or different when they occur more than once;
R², R³ are each independently hydrogen;
   C₁-C₁₈-alkyl whose carbon chain may be interrupted by one or more —O—, —S—, —CO—, —SO— and/or —SO₂— moieties and which may be mono- or polysubstituted by C₁-C₁₂-alkoxy, C₁-C₆-alkylthio, hydroxyl, mercapto, halogen, cyano, nitro and/or —COOR¹;
   aryl or hetaryl, to each of which may be fuzed further saturated or unsaturated 5- to 7-membered rings whose carbon skeleton may be interrupted by one or more —O—, —S—, —CO— and/or —SO₂— moieties, where the entire ring system may be mono- or polysubstituted by C₁-C₁₂-alkyl and/or the above radicals mentioned as substituents for alkyl;
A is a 5- to 9-membered ring which is bonded via a nitrogen atom and whose carbon chain may be interrupted by one or more —O—, —S—, —NR¹—, —CO— and/or —SO₂— moieties, to which one or two unsaturated or saturated, 4- to 3-membered rings may be fused, whose carbon chain may likewise be interrupted by these moieties and/or —N═, and the overall ring system may be mono- or polysubstituted by:
   hydroxyl, nitro, —NHR², carboxyl, —COOR², —CONR²R³ or —NR²COR³;
   C₁-C₃₀-alkyl whose carbon chain may be interrupted by one or more —O—, —S—, —NR¹—, —CO— and/or —SO₂— moieties and which may be mono- or polysubstituted by cyano, hydroxyl, nitro, C₁-₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
   C₅-C₈-cycloalkyl whose carbon skeleton may be interrupted by one or more —O—, —S— and/or —NR¹— moieties and/or which may be mono- or polysubstituted by C₁₋C₆-alkyl;
   aryl or hetaryl, each of which may be mono- or polysubstituted by C₁-C₁₈-alkyl, C₁-C₆-alkoxy, cyano, nitro, halogen, —CONR²R³, —NR²COR³, —SO₂N²R³ and/or aryl- or hetarylazo, each of which may be substituted by C₁-C₁₀-alkyl, C₁-C₆-alkoxy or cyano,
   and where the A radicals may be the same or different when m>1;
X is chlorine or bromine, where the X radicals may be the same or different when x>1;
Z is C₃-C₂₀-alkyl, C₃-C₂₀-alken-2-yl or C₃-C₂₀-alkyn-2-yl, whose alkyl chain may in each case be interrupted by one or more —O—, —S—, —NR¹—, —CO— and/or —SO₂— moieties and which may be mono- or polysubstituted by cyano, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
   aryloxy, arylthio, hetaryloxy or hetarylthio, to each of which may be fuzed further saturated or unsaturated 5- to 7-membered rings whose carbon skeleton may be interrupted by one or more —O—, —S—, —NR¹—, —N═CR¹—, —CO—, —SO— and/or —SO₂— moieties, where the entire ring system may be mono- or polysubstituted by the alkyl radicals (1), cycloalkyl radicals (2), aryl or hetaryl radicals (3) and/or the (i) and/or (iv) radicals mentioned there for R and R′,
   where the Z radicals may be the same or different when z>1;
m is 1 or 2;
n is from 1 to 4 when m=1;
   is from 1 to 6 when m=2;
x is from 0 to 3 when m=1, where n+x+z≦4,
   is from 0 to 5 when m=2, where n+x+z≦6;
z is from 0 to 3when m=1, where n+x+z≦4,
   is from 0 to 5 when m=2, where n+x+z≦6.

The invention further relates to the preparation of these rylenetetracarboximides and to their use for coloring organic and inorganic materials, in particular coating materials, printing inks and plastics, as dispersion assistants and pigment additives for organic pigments, for producing aqueous polymer dispersions which absorb in the near infrared region of the electromagnetic spectrum, for obtaining markings and inscriptions which absorb infrared light and are invisible to the human eye, and as infrared absorbers for heat management.

The higher rylenetetracarboximides (referred to as “rylenimides” for short below) are known to be of particular interest from application points of view owing to their strong absorption in the near infrared region of the electromagnetic spectrum. For example, WO-A-02/77081 describes the use of quaterrylenetetracarboximides as infrared absorbers for thermal protection in glass laminates.

In addition to the rylenimides unsubstituted in the rylene skeleton, terrylene- and quaterrylene-based rylenimides are known which bear halogen, aryloxy, arylthio, hetaryloxy, hetarylthio, alkyl, alkenyl or alkynyl as substituents on the rylene skeleton (Chem. Eur. J. 3, 219-225 (1997); WO-A-03/104232; WO-A-96/22332; Angew. Chem. 107, 1487-1489 (1995); WO-A-02/76988). Tetrahedron Letters 40, 7047-7050 (1999) discloses N,N′-dicyclohexylperylene-3,4:9,10-tetracarboximides which bear 2 pyrrolidinyl, piperidyl or morpholinyl radicals in the perylene skeleton.

It is an object of the invention to provide rylenimides having advantageous application properties which not only can be incorporated readily into the particular application media and are adaptable to these media, but also absorb at a longer wavelength than the rylenimides known to date.

Accordingly, the rylenimides of the formula I defined at the outset have been found.

Preferred and particularly preferred rylenimides I can be taken from the subclaims.

In addition, a process has been found for preparing rylenimides of the general formula Ia

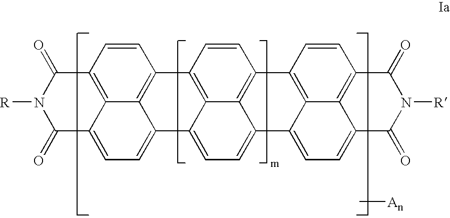

in which the variables are each as defined at the outset, which comprises reacting a halogenated rylenetetracarboximides of the formula IIa

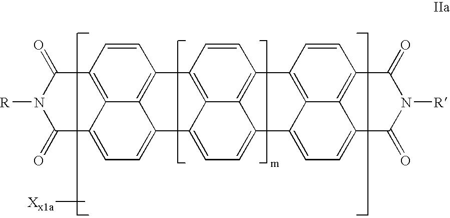

in which X and x1a are each defined as follows:
X is halogen;
x1a is from 1 to 4 when m=1;
   is from 1 to 6 when m=2,
      if desired in the presence of a nonacidic solvent, with a cyclic amine of the formula III

H-A III

or a salt of this amine.

In addition, a process has been found for preparing rylenimides of the general formula Ib

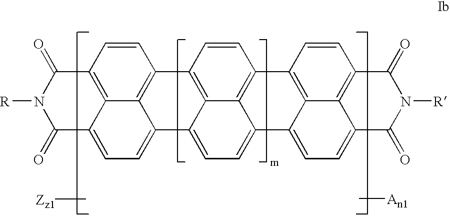

in which R, R′, A, Z and m are each as defined at the outset and n1 and z1 are defined as follows:
n1 is from 1 to 3 when m=1;
   is from 1 to 5 when m=2;
z1 is from 1 to 3 when m=1 where n1+z1≦4;
   is from 1 to 5 when m=2 where n1+z1≦6,
      which comprises reacting a rylenimide of the general formula IIb

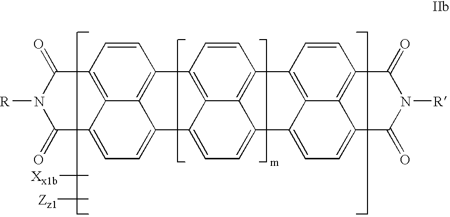

in which X and x1b are each defined as follows:
X is halogen;
x1b is from 1 to 3 when m=1 where x1b+z1≦4;
   is from 1 to 5 when m=2 where x1b+z1≦6,
      if desired in the presence of a nonacidic solvent, with a cyclic amine of the abovementioned formula III or a salt of this amine.

Finally, the use has been found of the rylenimides I for coloring high molecular weight organic and inorganic materials, as dispersion assistants and pigment additives for organic pigments, for producing aqueous polymer dispersions which absorb in the near infrared region of the electromagnetic spectrum, for obtaining markings which absorb infrared light and are invisible to the human eye and as infrared absorbers for heat management.

Specific examples of the R, R′, R¹, R², A, X and Z radicals mentioned in the formulae and their substituents are as follows:

methyl, ethyl, propyl, isopropyl, butyl, isobutyl, tert-butyl, pentyl, isopentyl, neopentyl, tert-pentyl, hexyl, 2-methylpentyl, heptyl, 1-ethylpentyl, octyl, 2-ethylhexyl, isooctyl, nonyl, isononyl, decyl, isodecyl, undecyl, dodecyl, tridecyl, isotridecyl, tetradecyl, pentadecyl, hexadecyl, heptadecyl, octadecyl, nonadecyl and eicosyl (the above terms isooctyl, isononyl, isodecyl and isotridecyl are trivial terms and stem from the alcohols obtained by the oxo process);

2-methoxyethyl, 2-ethoxyethyl, 2-propoxyethyl, 2-isopropoxyethyl, 2-butoxyethyl, 2- and 3-methoxypropyl, 2- and 3-ethoxypropyl, 2- and 3-propoxypropyl, 2- and 3-butoxypropyl, 2- and 4-methoxybutyl, 2- and 4-ethoxybutyl, 2- and 4-propoxybutyl, 3,6-dioxaheptyl, 3,6-dioxaoctyl, 4,8-dioxanonyl, 3,7-dioxaoctyl, 3,7-dioxanonyl, 4,7-dioxaoctyl, 4,7-dioxanonyl, 2- and 4-butoxybutyl, 4,8-dioxadecyl, 3,6,9-trioxadecyl, 3,6,9-trioxaundecyl, 3,6,9-trioxadodecyl, 3,6,9,12-tetraoxatridecyl and 3,6,9,12-tetra-oxatetradecyl;

2-methylthioethyl, 2-ethylthioethyl, 2-propylthioethyl, 2-isopropylthioethyl, 2-butylthioethyl, 2- and 3-methylthiopropyl, 2- and 3-ethylthiopropyl, 2- and 3-propylthiopropyl, 2- and 3-butylthiopropyl, 2- and 4-methylthiobutyl, 2- and 4-ethyl-thiobutyl, 2- and 4-propylthiobutyl, 3,6-dithiaheptyl, 3,6-dithiaoctyl, 4,8-dithianonyl, 3,7-dithiaoctyl, 3,7-dithianonyl, 2- and 4-butylthiobutyl, 4,8-dithiadecyl, 3,6,9-tri-thiadecyl, 3,6,9-trithiaundecyl, 3,6,9-trithiadodecyl, 3,6,9,12-tetrathiatridecyl and 3,6,9,12-tetrathiatetradecyl;

2-monomethyl- and 2-monoethylaminoethyl, 2-dimethylaminoethyl, 2- and 3-dimethyl-aminopropyl, 3-monoisopropylaminopropyl, 2- and 4-monopropylaminobutyl, 2- and 4-dimethylaminobutyl, 6-methyl-3,6-diazaheptyl, 3,6-dimethyl-3,6-diazaheptyl, 3,6-diazaoctyl, 3,6-dimethyl-3,6-diazaoctyl, 9-methyl-3,6,9-triazadecyl, 3,6,9-trimethyl-3,6,9-triazadecyl, 3,6,9-triazaundecyl, 3,6,9-trimethyl-3,6,9-triazaundecyl, 12-methyl-3,6,9,12-tetraazatridecyl and 3,6,9,12-tetramethyl-3,6,9,12-tetraazatridecyl;

(1-ethylethylidene)aminoethylene, (1-ethylethylidene)aminopropylene, (1-ethylethylidene)aminobutylene, (1-ethylethylidene)aminodecylene and (1-ethylethylidene)aminododecylene;

propan-2-on-1-yl, butan-3-on-1-yl, butan-3-on-2-yl and 2-ethylpentan-3-on-1-yl;

2-methylsulfoxidoethyl, 2-ethylsulfoxidoethyl, 2-propylsulfoxidoethyl, 2-isopropylsulfoxidoethyl, 2-butylsulfoxidoethyl, 2- and 3-methylsulfoxidopropyl, 2- and 3-ethylsulfoxidopropyl, 2- and 3-propylsulfoxidopropyl, 2- and 3-butylsulfoxidopropyl, 2- and 4-methylsulfoxidobutyl, 2- and 4-ethylsulfoxidobutyl, 2- and 4-propylsulfoxidobutyl and 4-butylsulfoxidobutyl;

2-methylsulfonylethyl, 2-ethylsulfonylethyl, 2-propylsulfonylethyl, 2-isopropylsulfonyl-ethyl, 2-butylsulfonylethyl, 2- and 3-methylsulfonylpropyl, 2- and 3-ethylsulfonylpropyl, 2- and 3-propylsulfonylpropyl, 2- and 3-butylsulfonylpropyl, 2- and 4-methylsulfonyl-butyl, 2- and 4-ethylsulfonylbutyl, 2- and 4-propylsulfonylbutyl and 4-butylsulfonylbutyl;

carboxymethyl, 2-carboxyethyl, 3-carboxypropyl, 4-carboxybutyl, 5-carboxypentyl, 6-carboxyhexyl, 8-carboxyoctyl, 10-carboxydecyl, 12-carboxydodecyl and 14-carboxy-tetradecyl;

sulfomethyl, 2-sulfoethyl, 3-sulfopropyl, 4-sulfobutyl, 5-sulfopentyl, 6-sulfohexyl, 8-sulfooctyl, 10-sulfodecyl, 12-sulfododecyl and 14-sulfotetradecyl;

2-hydroxyethyl, 2- and 3-hydroxypropyl, 1-hydroxyprop-2-yl, 3- and 4-hydroxybutyl, 1-hydroxybut-2-yl and 8-hydroxy-4-oxaoctyl;

2-cyanoethyl, 3-cyanopropyl, 3- and 4-cyanobutyl, 2-methyl-3-ethyl-3-cyanopropyl, 7-cyano-7-ethylheptyl and 4,7-dimethyl-7-cyanoheptyl;

2-chloroethyl, 2- and 3-chloropropyl, 2-,3- and 4-chlorobutyl, 2-bromoethyl, 2- and 3-bromopropyl and 2-,3- and 4-bromobutyl;

2-nitroethyl, 2- and 3-nitropropyl and 2-,3- and 4-nitrobutyl;

methoxy, ethoxy, propoxy, isopropoxy, butoxy, isobutoxy, sec-butoxy, tert-butoxy, pentoxy, isopentoxy, neopentoxy, tert-pentoxy and hexoxy;

methylthio, ethylthio, propylthio, isopropylthio, butylthio, isobutylthio, sec-butylthio, tert-butylthio, pentylthio, isopentylthio, neopentylthio, tert-pentylthio and hexylthio;

methylamino, ethylamino, propylamino, isopropylamino, butylamino, isobutylamino, pentylamino, hexylamino, dimethylamino, methylethylamino, diethylamino, dipropylamino, diisopropylamino, dibutylamino, diisobutylamino, dipentylamino, dihexylamino, dicyclopentylamino, dicyclohexylamino, dicycloheptylamino, diphenylamino and dibenzylamino;

formylamino, acetylamino, propionylamino and benzoylamino;

carbamoyl, methylaminocarbonyl, ethylaminocarbonyl, propylaminocarbonyl, butylaminocarbonyl, pentylaminocarbonyl, hexylaminocarbonyl, heptylaminocarbonyl, octylaminocarbonyl, nonylaminocarbonyl, decylaminocarbonyl and phenylaminocarbonyl;

aminosulfonyl, N,N-dimethylaminosulfonyl, N,N-diethylaminosulfonyl, N,N-dipropylaminosulfonyl, N,N-diisopropylaminosulfonyl, N,N-dibutylaminosulfonyl, N,N-diisobutylaminosulfonyl, N-N-di-sec-butylaminosulfonyl, N,N-di-tert-butylaminosulfonyl, N,N-dipentylaminosulfonyl, N,N-dihexylaminosulfonyl, N,N-diheptylaminosulfonyl, N,N-dioctylaminosulfonyl, N,N-dinonylaminosulfonyl, N,N-didecylaminosulfonyl, N,N-didodecylaminosulfonyl, N-methyl-N-ethylaminosulfonyl, N-methyl-N-dodecylaminosulfonyl, N-dodecylaminosulfonyl, (N,N-dimethylamino)ethylaminosulfonyl, N,N-(propoxyethyl)dodecylaminosulfonyl, N,N-diphenylaminosulfonyl, N,N-(4-tert-butylphenyl)octadecylaminosulfonyl and N,N-bis(4-chlorophenyl)aminosulfonyl; methoxycarbonyl, ethoxycarbonyl, propoxycarbonyl, isopropoxycarbonyl, hexoxycarbonyl, dodecyloxycarbonyl, octadecyloxycarbonyl, phenoxycarbonyl, (4-tert-butyl-phenoxy)carbonyl and (4-chlorophenoxy)carbonyl;

methoxysulfonyl, ethoxysulfonyl, propoxysulfonyl, isopropoxysulfonyl, butoxysulfonyl, isobutoxysulfonyl, tert-butoxysulfonyl, hexoxysulfonyl, dodecyloxysulfonyl, octadecyloxysulfonyl, phenoxysulfonyl, 1- and 2-naphthyloxysulfonyl, (4-tert-butylphenoxy)sulfonyl and (4-chlorophenoxy)sulfonyl;

chlorine, bromine and iodine;

phenylazo, 2-naphthylazo, 2-pyridylazo and 2-pyrimidylazo;

cyclopropyl, cyclobutyl, cyclopentyl, 2- and 3-methylcyclopentyl, 2- and 3-ethylcyclo-pentyl, cyclohexyl, 2-,3- and 4-methylcyclohexyl, 2-,3- and 4-ethylcyclohexyl, 3- and 4-propylcyclohexyl, 3- and 4-isopropylcyclohexyl, 3- and 4-butylcyclohexyl, 3- and 4-sec-butylcyclohexyl, 3- and 4-tert-butylcyclohexyl, cycloheptyl, 2-,3- and 4-methyl-cycloheptyl, 2-,3- and 4-ethylcycloheptyl, 3- and 4-propylcycloheptyl, 3- and 4-isopropylcycloheptyl, 3- and 4-butylcycloheptyl, 3- and 4-sec-butylcycloheptyl, 3- and 4-tert-butylcycloheptyl, cyclooctyl, 2-,3-,4- and 5-methylcyclooctyl, 2-,3-,4- and 5-ethylcyclooctyl and 3-,4- and 5-propylcyclooctyl; 3- and 4-hydroxycyclohexyl, 3- and 4-nitrocyclohexyl and 3- and 4-chlorocyclohexyl;

1-,2- and 3-cyclopentenyl, 1-,2-,3- and 4-cyclohexenyl, 1-2- and 3-cycloheptenyl and 1-,2-,3- and 4-cyclooctenyl;

2-dioxanyl, 1-morpholinyl, 1-thiomorpholinyl, 2- and 3-tetrahydrofuryl, 1-,2- and 3-pyrrolidinyl, 1-piperazyl, 1-diketopiperazyl and 1-,2-,3- and 4-piperidyl;

phenyl, 2-naphthyl, 2- and 3-pyrryl, 2-,3- and 4-pyridyl, 2-,4- and 5-pyrimidyl, 3-, 4- and 5-pyrazolyl, 2-,4- and 5-imidazolyl, 2-,4- and 5-thiazolyl, 3-(1,2,4-triazyl), 2-(1,3,5-triazyl), 6-quinaldyl, 3-,5-,6- and 8-quinolinyl, 2-benzoxazolyl, 2-benzothiazolyl, 5-benzothiadiazolyl, 2- and 5-benzimidazolyl and 1- and 5-isoquinolyl;

1-,2-,3-,4-,5-,6- and 7-indolyl, 1-,2-,3-,4-,5-,6- and 7-isoindolyl, 5-(4-methylisoindolyl), 5-(4-phenylisoindolyl), 1-,2-,4-,6-7- and 8-(1,2,3,4-tetrahydroisoquinolinyl), 3-(5-phenyl)-(1,2,3,4-tetrahydroisoquinolinyl), 5-(3-dodecyl-(1,2,3,4-tetrahydroisoquinolinyl), 1-,2-,3-,4-,5-,6-,7- and 8-(1,2,3,4-tetrahydroquinolinyl) and 2-,3-,4-,5-,6-,7- and 8-chromanyl, 2-,4- and 7-quinolinyl, 2-(4-phenylquinolinyl) and 2-(5-ethylquinolinyl);

2-,3- and 4-methylphenyl, 2,4-,3,5- and 2,6-dimethylphenyl, 2,4,6-trimethylphenyl, 2-,3- and 4-ethylphenyl, 2,4-,3,5- and 2,6-diethylphenyl, 2,4,6-triethylphenyl, 2-,3- and 4-propylphenyl, 2,4-,3,5- and 2,6-dipropylphenyl, 2,4,6-tripropylphenyl, 2-,3- and 4-isopropylphenyl, 2,4-,3,5- and 2,6-diisopropylphenyl, 2,4,6-triisopropylphenyl, 2-,3- and 4-butylphenyl, 2,4-,3,5- and 2,6-dibutylphenyl, 2,4,6-tributylphenyl, 2-,3- and 4-isobutylphenyl, 2,4-,3,5- and 2,6-diisobutylphenyl, 2,4,6-triisobutylphenyl, 2-,3- and 4-sec-butylphenyl, 2,4-,3,5- and 2,6-di-sec-butylphenyl and 2,4,6-tri-sec-butylphenyl; 2-,3- and 4-methoxyphenyl, 2,4-,3,5- and 2,6-dimethoxyphenyl, 2,4,6-trimethoxyphenyl, 2-,3- and 4-ethoxyphenyl, 2,4-,3,5- and 2,6-diethoxyphenyl, 2,4,6-triethoxyphenyl, 2-,3- and 4-propoxyphenyl, 2,4-,3,5- and 2,6-dipropoxyphenyl, 2-,3- and 4-isopropoxyphenyl, 2,4- and 2,6-diisopropoxyphenyl and 2-,3- and 4-butoxyphenyl; 2-,3- and 4-chlorophenyl and 2,4-,3,5- and 2,6-dichlorophenyl; 2-,3- and 4-hydroxyphenyl and 2,4-,3,5- and 2,6-dihydroxyphenyl; 2-,3- and 4-cyanophenyl; 3- and 4-carboxyphenyl; 3- and 4-carboxamidophenyl, 3- and 4-N-methylcarboxamidophenyl and 3- and 4-N-ethylcarboxamidophenyl; 3- and 4-acetylaminophenyl, 3- and 4-propionylaminophenyl and 3- and 4-butyrylaminophenyl; 3- and 4-N-phenylaminophenyl, 3- and 4-N-(o-tolyl)aminophenyl, 3- and 4-N-(m-tolyl)aminophenyl and 3- and 4-N-(p-tolyl)aminophenyl; 3- and 4-(2-pyridyl)aminophenyl, 3- and 4-(3-pyridyl)aminophenyl, 3- and 4-(4-pyridyl)aminophenyl, 3- and 4-(2-pyrimidyl)aminophenyl and 4-(4-pyrimidyl)aminophenyl;

4-phenylazophenyl, 4-(1-naphthylazo)phenyl, 4-(2-naphthylazo)phenyl, 4-(4-naphthylazo)phenyl, 4-(2-pyridylazo)phenyl, 4-(3-pyridylazo)phenyl, 4-(4-pyridylazo)phenyl, 4-(2-pyrimidylazo)phenyl, 4-(4-pyrimidylazo)phenyl and 4-(5-pyrimidylazo)phenyl;

phenoxy, phenylthio, 2-naphthoxy, 2-naphthylthio, 2-,3- and 4-pyridyloxy, 2-,3- and 4-pyridylthio, 2-,4- and 5-pyrimidyloxy and 2-,4- and 5-pyrimidylthio;

ethynyl, 1- and 2-propynyl, 1-,2- and 3-butynyl, 1-,2-,3- and 4-pentynyl, 3-methyl-1-butynyl, 1-,2-,3-,4- and 5-hexynyl, 3- and 4-methyl-1-pentynyl, 3,3-dimethyl-1-butynyl, 1-heptynyl, 3-,4- and 5-methyl-1-hexynyl, 3,3-,3,4- and 4,4-dimethyl-1-pentynyl, 3-ethyl-1-pentynyl, 1-octynyl, 3-,4-,5- and 6-methyl-1-heptynyl, 3,3-,3,4-,3,5-, 4,4- and 4,5-dimethyl-1-hexynyl, 3-,4- and 5-ethyl-1-hexynyl, 3-ethyl-3-methyl-1-pentynyl, 3-ethyl-4-methyl-1-pentynyl, 3,3,4- and 3,4,4-trimethyl-1-pentynyl, 1-nonynyl, 1-decynyl, 1-undecynyl and 1-dodecynyl;

4-cyano-1-butynyl, 5-cyano-1-pentynyl, 6-cyano-1-hexynyl, 7-cyano-1-heptynyl and 8-cyano-1-octynyl;

4-hydroxy-1-butynyl, 5-hydroxy-1-pentynyl, 6-hydroxy-1-hexynyl, 7-hydroxy-1-heptynyl, 8-hydroxy-1-octynyl, 9-hydroxy-1-nonynyl, 10-hydroxy-1-decynyl, 11-hydroxy-1-undecynyl and 12-hydroxy-1-dodecynyl;

4-carboxy-1-butynyl, 5-carboxy-1-pentynyl, 6-carboxy-1-hexynyl, 7-carboxy-1-heptynyl, 8-carboxy-1-octynyl, 4-methylcarboxy-1-butynyl, 5-methylcarboxy-1-pentynyl, 6-methylcarboxy-1-hexynyl, 7-methylcarboxy-1-heptynyl, 8-methylcarboxy-1-octynyl, 4-ethylcarboxy-1-butynyl, 5-ethylcarboxy-1-pentynyl, 6-ethylcarboxy-1-hexynyl, 7-ethylcarboxy-1-heptynyl and 8-ethylcarboxy-1-octynyl;

1ethenyl, 1- and 2-propenyl, 1-,2- and 3-butenyl, 1-,2-,3- and 4-pentenyl, 3-methyl-1-butenyl, 1-,2-,3-,4- and 5-hexenyl, 3- and 4-methyl-1-pentenyl, 3,3-dimethyl-1-butenyl, 1-heptenyl, 3-,4- and 5-methyl-1-hexenyl, 3,3-,3,4- and 4,4-dimethyl-1-pentenyl, 3-ethyl-1-pentenyl, 1-octenyl, 3-,4-,5- and 6-methyl-1-heptenyl, 3,3-,3,4-,3,5-,4,4- and 4,5-dimethyl-1-hexenyl-3-,4- and 5-ethyl-1-hexenyl, 3-ethyl-3-methyl-1-pentenyl, 3-ethyl-4-methyl-1-pentenyl, 3,3,4- and 3,4,4-trimethyl-1-pentenyl, 1-nonenyl, 1-decenyl, 1-undecenyl and 1-dodecenyl;

4-cyano-1-butenyl, 5-cyano-1-pentenyl, 6-cyano-1-hexenyl, 7-cyano-1-heptenyl and 8-cyano-1-octenyl;

4-hydroxy-1-butenyl, 5-hydroxy-1-pentenyl, 6-hydroxy-1-hexenyl, 7-hydroxy-1-heptenyl, 8-hydroxy-1-octenyl, 9-hydroxy-1-nonenyl, 10-hydroxy-1-decenyl, 11-hydroxy-1-undecenyl and 12-hydroxy-1-dodecenyl;

4-carboxy-1-butenyl, 5-carboxy-1-pentenyl, 6-carboxy-1-hexenyl, 7-carboxy-1-heptenyl, 8-carboxy-1-octenyl, 4-methylcarboxy-1-butenyl, 5-methylcarboxy-1-pentenyl, 6-methylcarboxy-1-hexenyl, 7-methylcarboxy-1-heptenyl, 8-methylcarboxy-1-octenyl, 4-ethylcarboxy-1-butenyl, 5-ethylcarboxy-1-pentenyl, 6-ethylcarboxy-1-hexenyl, 7-ethylcarboxy-1-heptenyl and 8-ethylcarboxy-1-octenyl.

The inventive terrylenimides I may bear up to 6 substituents in the terrylene skeleton; preference is given to 4 or 2 substituents. The inventive quaterrylenimides I may have up to 8 substituents in the quaterrylene skeleton; preference is given to 6, 4 or 2 substituents.

The rylene skeletons are preferably substituted by at least 2 cyclic amino radicals A which, as a constituent of the chromophoric system, bring about an unexpectedly strong bathochromic shift of absorption and emission which is about 110 nm in the case of the terrylenimides and about 60 nm in the case of the quaterrylinimides compared to the rylenimides which are in each case unsubstituted or substituted by other radicals.

The inventive rylenimides are obtained in the preparation generally in the form of product mixtures having a differing degree of substitution, which may be separated by chromatography if desired.

The inventive rylenimides Ia which are substituted exclusively by the cyclic amino radicals A may advantageously be prepared by reacting the halogenated, preferably the chlorinated and more preferably the brominated, rylenimides IIa with the corresponding amine III.

The halogenated rylenimides IIa and the preparation thereof are known from WO-A-03/104232, 96/22332 and 02/76988, and from the prior German patent application 102004048729.4.

The rylenimides Ib which are substituted in the rylene skeleton both by the cyclic amine radicals A and by (het)aryloxy and/or (het)arylthio radicals Z and are likewise in accordance with the invention may be prepared starting from rylenimides IIb which have already been substituted by the Z radicals and bear further halogen atoms X for the substitution by the cyclic amino radicals A.

The rylenimides IIb are likewise known from the abovementioned WO-A-03/104232, 96/22332 and 02/76988 and are obtainable from the halogenated rylenimides IIa by incomplete substitution of the halogen atoms X by the Z radicals.

Inventive rylenimides I which bear cyclic amino radicals A, (het)aryloxy and/or (het)arylthio radicals Z and halogen atoms X or cyclic amino radicals A and halogen atoms X in the rylene skeleton are obtainable analogously by in each case incomplete substitution of the halogen atoms X by the amino radicals A and, if appropriate, the Z radicals. However, these rylenimides I are only of minor importance.

In the inventive reaction of the rylenimide IIa or IIb with the cyclic amine III, a nonacidic solvent may be present as a reaction medium, but the amine III may itself also function as a solvent.

Suitable cyclic amines III are in particular piperidines, pyrrolidines, piperazines, morpholines and thiomorpholines (1,4-thiazines), of which preference is given to the piperidines, pyrrolidines, piperazines and morpholines, and particular preference to the piperidines.

The cyclic amines III may be chemically modified, i.e. their carbon chain may be interrupted not only by —O—, —S— or —NR¹—, but also by —CO—, —SO— or —SO₂—, they may have one or two aromatic or saturated 4- to 7-membered fused rings whose carbon chain may likewise be interrupted by the moieties mentioned, and they may be substituted by the alkyl, cycloalkyl and/or (het)aryl radicals mentioned at the outset. However, preference is given to the unmodified amines III.

Specific examples of suitable amines III are as follows: piperidine, 2- or 3-methylpiperidine, 6-ethylpiperidine, 2,6- or 3,5-dimethylpiperidine, 2,2,6,6-tetramethylpiperidine, 4-benzylpiperidine, 4-phenylpiperidine, piperidin-4-ol, piperidine-4-carboxylic acid, methyl piperidine-4-carboxylate, ethyl piperidine-4-carboxylate, piperidine-4-carboxamide, 2,2,6,6-tetramethylpiperidin-4-one, 2,2,6,6-tetramethylpiperidin-4-ylamine, decahydroquinoline and decahydroisoquinoline;

pyrrolidine, 2-methylpyrrolidine, 2,5-dimethylpyrrolidine, 2,5-diethylpyrrolidine, tropanol, methyl pyrrolidine-2-carboxylate, ethyl pyrrolidine-2-carboxylate, benzyl pyrrolidine-2-carboxylate, pyrrolidine-2-carboxamide, 2,2,5,5-tetramethylpyrrolidine-3-carboxylic acid, methyl 2,2,5,5-tetramethylpyrrolidine-3-carboxylate, ethyl 2,2,5,5-tetramethylpyrrolidine-3-carboxylate, benzyl 2,2,5,5-tetramethylpyrrolidine-3-carboxylate, pyrrolidin-3-ylamine, (2,6-dimethylphenyl)pyrrolidin-2-ylmethylamine, (2,6-diisopropylphenyl)pyrrolidin-2-ylmethylamine and dodecahydrocarbazole;

piperazine, diketopiperazine;, 1-benzylpiperazine, 1-phenethylpiperazine, 1-cyclohexyl-piperazine, 1-phenylpiperazine, 1-(2,4-dimethylphenyl)piperazine, 1-(2-,3- or 4-methoxyphenyl)piperazine, 1-(2-,3- or 4-ethoxyphenyl)piperazine, 1-(2-,3- or 4-fluorophenyl)piperazine, 1-(2-,3- or 4-chlorophenyl)piperazine, 1-(2-,3- or 4-bromophenyl)-piperazine, 1-,2- or 3-pyridin-2-ylpiperazine and 1-benzo[1,3]dioxol-4-ylmethyl-piperazine;

morpholine, 2,6-dimethylmorpholine, 3,3,5,5-tetramethylmorpholine, morpholin-2- or -3-ylmethanol, morpholin-2- or -3-ylacetic acid, methyl morpholin-2- or -3-ylacetate, ethyl morpholin-2- or -3-ylacetate, methyl 3-morpholin-3-ylpropionate, ethyl 3-morpholin-3-ylpropionate, tert-butyl 3-morpholin-3-ylpropionate, morpholin-2- or -3-ylacetamide, 3-morpholin-3-yl-propionamide, 3-benzylmorpholine, 3-methyl-2-phenylmorpholine, 2- or 3-phenylmorpholine, 2-(4-methoxyphenyl)morpholine, 2-(4-trifluoromethylphenyl)-morpholine, 2-(4-chlorophenyl)morpholine, 2-(3,5-dichlorophenyl)morpholine, morpholine-2- or -3-carboxylic acid, methyl morpholine-3-carboxylate, 3-pyridin-3-ylmorpholine, 5-phenylmorpholin-2-one, 2-morpholin-2-ylethylamine and phenoxazine;

thiomorpholine, 2- or 3-phenylthiomorpholine, 2- or 3-(4-methoxyphenyl)thiomorpholine, 2- or 3-(4-fluorophenyl)thiomorpholine, 2- or 3-(4-trifluoromethylphenyl)-thiomorpholine, 2- or 3-(2-chlorophenyl)thiomorpholine, 4-(2-aminoethyl)thiomorpholine, 3-pyridin-3-ylthiomorpholine, 3-thiomorpholinecarboxylic acid, 6,6-dimethyl-5-oxo-3-thiomorpholinecarboxylic acid, 3-thiomorpholinone and 2-phenylthiomorpholin-3-one, and the thiomorpholine oxides and dioxides.

Instead of the free amines III, it is also possible to use salts thereof, for example the salts of inorganic acids such as the hydrofluorides, hydrochlorides, hydrobromides, hydroiodides, hydrogensulfates, hydrogensulfites, hydrogenphosphates and hydrogenphosphites, or the salts or organic acids such as the formates, acetates and propionates, from which the amines can be released again by base addition.

When the cyclic amine III is used only as a reaction partner and not simultaneously as a solvent, its use amount is typically from 1 to 10 mol, in particular from 1 to 3 mol, per halogen atom in the rylenimide IIa or IIb to be substituted.

The amount of cyclic amine III may, if desired, be reduced virtually to the stoichiometrically required amount by adding a non-nucleophilic nitrogen base. Bases include trialkylamines which are liquid under the reaction conditions, in particular tri(C₃-C₆-alkyl)amines such as tripropylamine and tributylamine, and in particular nitrogen heterocycles such as pyridine, N-methylpiperidine, N-methylmorpholine, pyrimidine, quinoline, isoquinoline, quinaldine, diazabicyclononene (DBN) and diazabicyclo-undecene (DBU).

When a non-nucleophilic base is used, its amount is generally from 1 to 5 mol, preferably from 1 to 3 mol, per halogen atom in the rylenimide IIa or IIb to be substituted.

Suitable solvents for the inventive reaction of the halogenated rylenimide IIa or IIb with the cyclic amine III are nonacidic solvents which do not protonate the amine III.

One group of preferred solvents is that of aliphatic carboxamides, in particular N,N-di(C₁-C₆-alkyl)-C₁-C₆carboxamides such as N,N-dimethylformamide, N,N-diethylformamide, N,N-dimethylacetamide and N,N-dimethylbutyramide, and lactams, in particular N—(C₁-C₆-alkyl)-lactams, such as N-methylpyrrolidone, of which particular preference is given to dimethylformamide and N-methylpyrrolidone.

A further group of preferred solvents is that of halogenated aromatic and aliphatic hydrocarbons such as chlorobenzene, dichlorobenzene and trichlorobenzene, and also halogenated methanes and ethanes, e.g. methylene chloride, chloroform, tribromomethane, tetrachloromethane, tetrabromomethane, 1,2-dichloro-, 1,1- and 1,2-dibromo-, 1,1,1- and 1,1,2-trichloro-, 1,1,1- and 1,1,2-tribromo-, 1,1,1,2- and 1,1,2,2-tetrachloro- and 1,1,1,2- and 1,1,2,2-tetrabromoethane, of which particular preference is given to methylene chloride, chloroform and chlorobenzene.

In addition, it would also be possible to use solvents which dissolve only the amine III but not the halogenated rylenimid IIa. In addition to protic solvents which do not protonate the amine III, such as water, aliphatic C₁-C₆-alcohols and glycols of the HO—(C₂H₄O)ₐ-H type where a=from 1 to 6, mention should be made of the di(C₁-C₆-alkyl)ethers of these glycols and aromatic hydrocarbons as suitable examples of these solvents.

The amount of solvent in the process according to the invention is selected in such a way that generally from 10 to 100 g, preferably from 10 to 50 g, of a phase which is liquid under the reaction conditions are present per g of halogenated rylenimide IIa or IIb.

The liquid phase is composed of the nonacidic solvent and the cyclic amine III or, if the cyclic amine III functions simultaneously as a solvent, of the amine III alone and, if appropriate, the non-nucleophilic base.

The reaction temperature in the process according to the invention is generally from 30 to 200° C., preferably from 50 to 150° C.

If the selected reaction temperature is above the boiling point of one of the components, it is possible to work in a closed system under the pressure which becomes established.

The reaction time is typically from 12 h to 10 d, in particular from 1 to 5 d.

In process technology terms, the procedure in accordance with the invention may be to heat a mixture of rylenimide IIa or IIb and cyclic amine III and, if desired, nonacidic solvent and/or non-nucleophilic base to the selected reaction temperature and stir it at this temperature for from 12 h to 10 d.

The workup of the reaction mixture the rylenimides I may be effected by filtering off any reaction product which has precipitated out or concentrating by evaporation, and subsequent column filtration or column chromatography.

Suitable substances for precipitating the reaction product are protic solvents such as water, alcohols, in particular C₁-C₆-alkanols, e.g. methanol, ethanol, n- and i-propanol, n-, i- and sec-butanol, n-pentanol, amyl alcohol and n- and i-hexanol, ethylene glycol mono(C₁-C₄-alkyl)ether, e.g. ethylene glycol mono-n-butyl ether, and carboxylic acids, in particular aliphatic C₁-C₄-carboxylic acids, e.g. formic acid, acetic acid, propionic acid and butyric acid.

The reaction product which is subsequently isolated by filtration or by evaporative concentration is typically washed with one of these solvents or a dilute inorganic acid such as sulfuric acid, hydrochloric acid or phosphoric acid, or a solvent combination, or else stirred and filtered again in these solvents.

In addition, a recrystallization from the chlorinated hydrocarbons or carboxamides and lactams specified as reaction solvents, if appropriate in combination with the solvents or dilute inorganic acids suitable for the precipitation, may be undertaken.

The final column chromatography allows the reaction product to be further purified. In addition, the rylenimides I of different degrees of substitution can be separated from one another.

Suitable eluents are the abovementioned chlorinated hydrocarbons, of which preference is given to chloroform and methylene chloride, and mixtures of esters, e.g. ethyl acetate, and/or alcohols, e.g. methanol or ethanol, and aliphatic hydrocarbons, e.g. petroleum ethers with boiling ranges of from 35 to 120° C., or aromatic hydrocarbons, e.g. benzene, toluene, xylenes, mesitylene and ethylbenzene. The stationary phase used is typically silica gel.

With the aid of the process according to the invention it is possible to obtain the rylenimides I in yields of generally from 30 to 90% and purities of at least 90%.

The inventive rylenimides I exhibit strong absorption in the near infrared region at wavelengths of up to 1100 nm and thus advantageously supplement the spectral region accessible with the aid of the rylene compounds known to date.

They are suitable for a multitude of applications, such as the coloring of high molecular weight organic and inorganic materials, for example of coating materials, printing inks and plastics, as dispersion assistants and pigment additives for organic pigments, for producing aqueous polymer dispersions which absorb in the near infrared region of the electromagnetic spectrum, for obtaining markings and inscriptions which absorb infrared light and are invisible to the human eye, and as infrared absorbers for heat management.

### EXAMPLES

### Example 1

### N,N′-(2,6-Diisopropyl)phenyl-1,6,9,13-tetra-(N-piperidyl)terrylene-3,4:11,12-tetracarboximide

A mixture of 4.6 g of N,N′-(2,6-diisopropyl)phenyl-1,6,9,13-tetrabromoterrylene-3,4:11,12-tetracarboximide and 60 ml of piperidine was stirred at 106° C. for 5 d. After the reaction mixture had been cooled to room temperature, the resulting product was filtered off, stirred first with 50 ml of ethanol, then with 100 ml of water and finally again with 50 ml of ethanol, in each case for 1 h, then filtered off and dried under reduced pressure.

Column filtration on silica gel with petroleum ether/ethyl acetate (8:1 v/v) afforded 3.9 g of a black solid (yield approx. 90%) which, according to mass spectroscopy analysis, corresponded to a mixture of N,N′-(2,6-diisopropylphenyl)terrylene-3,4:11,12-tetracarboximides bearing from 2 to 4 piperidyl radicals and exhibited the following absorption:

λₘₐₓ(ε)(methylene chloride)=791 nm(53 l g⁻¹ cm⁻¹).

In column chromatography with silica gel with chloroform, 1.5 g of pure N,N′-(2,6-diisopropylphenyl)-1,6,9,13-tetra-(N-piperidyl)terrylene-3,4:11,12-tetracarboximide were obtained (34% yield), which exhibited the following absorption:

λₘₐₓ(ε)(acetone)=538(4099), 804(21 582)nm(l mol⁻¹cm⁻¹);

λₘₐₓ(ε)(chloroform)=819 nm(23 000 l mol⁻¹cm⁻¹).

### Example 2

### Mixture of N,N′-(2,6-diisopropylphenyl)quaterrylene-3,4:13,14-tetracarboximides having from 1 to 6 piperidyl radicals in the quaterrylene skeleton

A mixture of 2.9 g of N,N′-(2,6-diisopropylphenyl)hexabromoquaterrylene-3,4:13,14-tetracarboximide, 30 ml of piperidine and 30 ml of dimethylformamide was stirred under reflux (106° C.) for 5 d.

After cooling to room temperature, the reaction product was precipitated by adding 10 ml of water and 10 ml of ethanol, filtered off, stirred repeatedly with 50 ml of water at 80° C. and filtered off, and finally washed three times with water/ethanol (1:1 v/v) and then dried under reduced pressure.

After column filtration on silica gel with toluene/ethyl acetate (10:1 v/v), 0.96 g of a black solid was obtained (yield approx. 40%), which, according to mass spectroscopy analysis, corresponded to a mixture of N,N′-(2,6-diisopropylphenyl)terrylene-3,4:11,12-tetracarboximides bearing from 1 to 6 piperidyl radicals (position of the piperidyl radicals in the hexasubstituted diimide: 1,6,8,11,16,19 and 1,6,8,11,16,18) and exhibited the following absorption:

λₘₐₓ(ε)(methylene chloride)=805 nm(61 l g⁻¹ cm⁻¹).

### Example 3

### N,N′-(2,6-Diisopropylphenyl)-1,6,11,16-tetra(4-tert-octylphenoxy)-8,18-di(N-piperidyl)-quaterrylene-3,4:13,14-tetracarboximide

A mixture of 1.94 g of an isomer mixture comprising N,N′-(2,6-diisopropylphenyl)-1,6,1,16-tetra(4-tert-octylphenoxy)-8, -and -8,19-dibromoquaterrylene-3,4:13,14-tetracarboximide (about 1:1) and 20 ml of piperidine was stirred at 85° C. for 5 d.

The reaction mixture was then introduced into a mixture of 100 ml of water and 100 ml of methanol and stirred at room temperature for 30 min before filtration. The moist presscake was stirred in 50 ml of 1M hydrochloric acid for 30 min, filtered off, then stirred in 100 ml of water/methanol (1:1 v/v), filtered off and dried under reduced pressure.

1.86 g of a black solid were obtained and were purified by column filtration on silica gel with toluene, 1.37 g of a black solid were obtained (70% yield) which, according to mass spectroscopy analysis, corresponded to a mixture of tetra(tert-octylphenoxy)-dipiperidyl, tetra(tert-octylphenoxy)monopiperidyl-, tri(tert-octylphenoxy)dipiperidyl- and tri(tert-octylphenoxy)tripiperidyl-substituted N,N′-(2,6-diisopropylphenyl)-quaterrylene-3,4:13,14-tetracarboximides, and exhibited the following absorption:

λₘₐₓ(ε)(methylene chloride)=847(40)nm(g mol⁻¹cm⁻¹).

In column chromatography on silica gel with methylene chloride, 1.1 g of an isomer mixture comprising N,N′-(2,6-diisopropylphenyl)-1,6,11,16-tetra(4-tert-octylphenoxy)-8,18 and -8,19-di(N-piperidyl)quaterrylene-3,4:13,14-tetracarboximide (about 1:1) were obtained (55% yield), which exhibited the following absorption:

λₘₐₓ(ε)(acetone)=489(13 015), 878(75 000)nm(l mol⁻¹cm⁻¹);

λₘₐₓ(ε)(chloroform)=910 nm(105 000 l mol⁻¹cm⁻¹).

### Example 4

### Mixture of N,N′-(2,6-diisopropylphenyl)quaterrylene-3,4:13,14-tetracarboximide with from 3 to 5 piperidyl radicals in the quaterrylene structure

A mixture of 20.23 g of N,N′-(2,6-diisopropylphenyl)hexabromoquaterrylene-3,4:13,14-tetracarboximide, 210 ml of piperidine and 210 ml of N-methylpyrrolidone was heated to 117° C. with stirring for 122 h.

After cooling to 80° C., the reaction mixture was precipitated in 500 ml of water. The mixture was stirred at 80° C. for 3 h. The precipitated reaction product was filtered off, washed with warm water and then dried under reduced pressure.

After column filtration on silica gel with toluene/ethyl acetate (10:1 v/v), 3.23 g of a black solid were obtained (yield approx. 42%), which, according to mass spectroscopy analysis, corresponded to a mixture of N,N′-2,6-diisopropylphenyl)terrylene-3,4:11,12-tetracarboximides bearing from 3 to 5 piperidyl radicals and exhibited the following absorption:

λₘₐₓ(ε)(methylene chloride)=877 nm(51 l g⁻¹cm⁻¹).

1. A rylenetetracarboximide of the general formula I 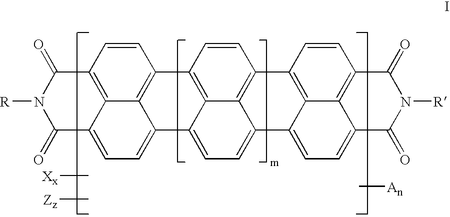
in which the variables are each defined as follows:
R, R′ are each independently:
hydrogen;
(1) C₁-C₃₀-alkyl whose carbon chain may be interrupted by one or more —O—, —S—, —NR¹—, —N═CR¹—, —C≡C—, —CR¹═CR¹—, —CO—, —SO— and/or —SO₂— moieties and which may be mono- or polysubstituted by:
(i) C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR² and/or —SO₃R²;
(ii) aryl or hetaryl, to which may be fuzed further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be mono- or polysubstituted by: C₁-C₁₈-alkyl, C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR², —SO₃R², aryl and/or hetaryl, each of which may be substituted by C₁-C₁₈-alkyl, C₁-C₁₂-alkoxy, hydroxyl, mercapto, halogen, cyano, nitro, —NR²R³, —NR²COR³, —CONR²R³, —SO₂NR²R³, —COOR² and/or —SO₃R²;
(iii) C₃-C₈-cycloalkyl whose carbon skeleton may be interrupted by one or more —O—, —S—, —NR¹—, —N═CR¹—, —CR¹═CR¹—, —CO—, —SO— and/or —SO₂— moieties and to which may be fuzed further saturated or unsaturated 5- to 7-membered rings whose carbon skeleton may be interrupted by one or more —O—, —S—, —NR¹—, —NR═CR¹—, —CR¹═CR¹—, —CO—, —SO— and/or —SO₂— moieties, where the entire ring system may be mono- or polysubstituted by: C₁-C₁₈-alkyl, C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR², and/or —SO₃²;
iv) a —U-aryl radical which may be mono- or polysubstituted by the above radicals mentioned as substituents for the aryl radicals (ii), where U is an —O—, —S—, —NR¹—, —CO—, —SO— or —SO₂— moiety;
(2) C₃-C₈-cycloalkyl to which may be fuzed further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be singly or multiply substituted by: the (i), (ii), (iii), (iv) radicals and/or
(v) C₁-C₃₀-alkyl whose carbon chain may be interrupted by one or more —O—, —S—, —NR¹—, —N═CR¹—, —C≡C—, —CR¹═CR¹—, —CO—, —SO— and/or —SO₂— moieties and which may be mono- or polysubstituted by: C₁C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR² and/or —SO₃R², aryl and/or saturated or unsaturated C₄-C₇-cycloalkyl whose carbon skeleton may be interrupted by one or more —O—, —S—, —NR¹—, —N═CR¹—, —CR¹═CR¹—, —CO—, —SO— and/or —SO₂— moieties, where the aryl and cycloalkyl radicals may each be mono- or polysubstituted by C₁-C₁₈-alkyl and/or the above radicals mentioned as substituents for alkyl;
(3) aryl or hetaryl to which may be fuzed further saturated or unsaturated 5- to 7-membered rings whose carbon skeleton may be interrupted by one or more —O—, —S—, —NR¹—, —N═CR¹—, —C≡C—, —CR¹═CR¹—, —CO—, —SO— and/or —SO₂— moieties, where the entire ring system may be substituted by the (i), (ii), (iii), (iv), (v) radicals and/or aryl- and/or hetarylazo, each of which may be substituted by C₁-C₁₀-alkyl, C₁-C₆-alkoxy and/or cyano;
R¹ is hydrogen or C₁-C₁₈-alkyl, where the R¹ radicals may be the same or different when they occur more than once;
R², R³ are each independently hydrogen;
C₁-C₁₈-alkyl whose carbon chain may be interrupted by one or more —O—, —S—, —CO—, —SO— and/or —SO₂— moieties and which may be mono- or polysubstituted by C₁-C₁₂-alkoxy, C₁-C₆-alkylthio, hydroxyl, mercapto, halogen, cyano, nitro and/or —COOR¹;
aryl or hetaryl, to each of which may be fuzed further saturated or unsaturated 5- to 7-membered rings whose carbon skeleton may be interrupted by one or more —O—, —S—, —CO— and/or —SO₂— moieties, where the entire ring system may be mono- or polysubstituted by C₁-C₁₂-alkyl and/or the above radicals mentioned as substituents for alkyl;
A is a 5- to 9-membered ring which is bonded via a nitrogen atom and whose carbon chain may be interrupted by one or more —O—, —S—, —NR¹—, —CO— and/or —SO₂— moieties, to which one or two unsaturated or saturated. 4- to 8-membered rings may be fused, whose carbon chain may likewise be interrupted by these moieties and/or —N═, and the overall ring system may be mono- or polysubstituted by:
hydroxyl, nitro, —NHR², carboxyl, —COOR², —CONR²R³ or —NR²COR³;
C₁-C₃₀-alkyl whose carbon chain may be interrupted by one or more —O—, —S—, —NR¹—, —CO— and/or —SO₂— moieties and which may be mono- or polysubstituted by cyano, hydroxyl, nitro, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
C₅-C₈-cycloalkyl whose carbon skeleton may be interrupted by one or more —O—, —S— and/or —NR¹— moieties and/or which may be mono- or polysubstituted by C₁-C₆-alkyl;
aryl or hetaryl, each of which may be mono- or polysubstituted by C₁-C₁₈-alkyl, C₁-C₆-alkoxy, cyano, nitro, halogen, —CONR²R³, —NR²COR³, —SO₂NR²R³ and/or aryl- or hetarylazo, each of which may be substituted by C₁-C₁₀-alkyl, C₁-C₆-alkoxy or cyano,
and where the A radicals may be the same or different when m>1;
X is chlorine or bromine, where the X radicals may be the same or different when x>1;
Z is C₃-C₂₀-alkyl, C₃-C₂₀-alken-2-yl or C₃-C₂₀-alkyn-2-yl, whose alkyl chain may in each case be interrupted by one or more —O—, —S—, —NR¹—, —CO— and/or —SO₂— moieties and which may be mono- or polysubstituted by cyano, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
aryloxy, arylthio, hetaryloxy or hetarylthio, to each of which may be fuzed further saturated or unsaturated 5- to 7-membered rings whose carbon skeleton may be interrupted by one or more —O—, —S—, —NR¹—, —N═CR¹—, —CO—, —SO— and/or —SO₂— moieties, where the entire ring system may be mono- or polysubstituted by the alkyl radicals (1), cycloalkyl radicals (2), aryl or hetaryl radicals (3) and/or the (i) and/or (iv) radicals mentioned there for R and R′,
where the Z radicals may be the same or different when z>1;
m is 1 or 2;
n is from 1 to 4 when m=1;
is from 1 to 6 when m=2;
x is from 0 to 3 when m=1, where n+x+z≦4,
is from 0 to 5 when m=2, where n+x+z≦6; and
z is from 0 to 3 when m=1, where n+x+z≦4,
is from 0 to 5 when m=2, where n+x+z≦6.

2. The rylenetetracarboximide of general formula I according to claim 1, in which the variables are each defined as follows:
R, R′ are each independently hydrogen;
C₁-C₃₀-alkyl whose carbon chain may interrupted by one or more —O—, —S—, —NR¹—, —CO— and/or —SO₂— moieties and which may be mono- or polysubstituted by cyano, C₁-C₆-alkoxy, aryl which max be substituted by C₁-C₁₈-alkyl or C₁-C₆-alkoxy, and/or a 5- to 7-membered heterocyclic radical which is bonded via a nitrogen atom and may comprise further heteroatoms and be aromatic;
C₅-C₈-cycloalkyl whose carbon skeleton may be interrupted by one or more —O—, —S— and/or —NR¹— moieties and/or which may be mono- or polysubstituted by C₁-C₆-alkyl;
aryl or hetaryl, each of which may be mono- or polysubstituted by C₁-C₁₈-alkyl, C₁-C₆-alkoxy, cyano, nitro, halogen, —CONR²R³, —SO₂NR²R³, —COOR², —SO₃R² and/or aryl- or hetarylazo, each of which may be substituted by C₁-C₁₀-alkyl, C₁-C₆-alkoxy or cyano,
R¹ is hydrogen or C₁-C₆-alkyl;
R², R³ are each independently hydrogen;
C₁-C₁₈-alkyl which may be substituted by C₁-C₆-alkoxy, halogen, hydroxyl, carboxyl and/or cyano; aryl or hetaryl, each of which may be substituted by the aforementioned radicals specified for alkyl and by C₁-C₆-alkyl;
A is a 5- to 9-membered ring which is bonded via a nitrogen atom and whose carbon chain may be interrupted by one more —O—, —S—, —NR¹—, —CO— and/or —SO₂— moieties, to which one or two unsaturated or saturated, 4- to 8-membered rings may be fused, whose carbon chain may likewise be interrupted by these moieties and/or —N═, and the overall ring system may be mono- or polysubstituted by:
hydroxyl, nitro, —NR²R³, —COOR², —CONR²R³ or —NR²COR³;
C₁-C₃₀-alkyl whose carbon chain may be interrupted by one or more —O—, —S—, —NR¹—, —CO— and/or —SO₂— moieties and which may be mono- or polysubstituted by cyano, hydroxyl, nitro, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
C₅-C₈-cycloalkyl whose carbon skeleton may be interrupted by one or more —O—, —S— and/or —NR¹— moieties and/or which may be mono- or polysubstituted by C₁-C₆-alkyl;
aryl or hetaryl, each of which may be mono- or polysubstituted by C₁-C₁₈-alkyl, C₁-C₆-alkoxy, cyano, nitro, halogen, —CONR²R³, —NR²COR³, —SO₂NR²R³ and/or aryl- or hetarylazo, each of which may be substituted by C₁-C₁₀-alkyl, C₁-C₆-alkoxy or cyano,
and where the A radicals may be the same or different when m>1;
X is chlorine or bromine, where the X radicals may be the same or different when x>1;
Z is C₃-C₂₀-alkyl, C₃-C₂₀-alken-2-yl or C₃-C₂₀-alkyn-2-yl, whose alkyl chain may in each case be interrupted by one or more —O—, —S—, —NR¹—, —CO— and/or —SO₂— moieties and which may be mono- or polysubstituted by cyano, C₁-C₆-alkoxy, —COOR², —CONR²R³, aryl which may be substituted by C₁-C₁₈-alkyl or C₁-C₆-alkoxy and/or a 5- to 7-membered heterocyclic radical which is bonded via a nitrogen atom and may comprise further heteroatoms and be aromatic;
aryloxy, arylthio, hetaryloxy or hetarylthio, to each of which may be fuzed further saturated or unsaturated 5- to 7-membered rings whose carbon skeleton may be interrupted by one or more —O—, —S—, —NR¹—, —N═CR¹—, —CO—, —SO— and/or —SO₂— moieties, where the entire ring system may be mono- or polysubstituted by the alkyl radicals (1), cycloalkyl radicals (2), aryl or hetaryl radicals (3) and/or the (i) and/or (iv) radicals mentioned there for R and R′,
where the Z radicals may be the same or different when z>1;
m is 1 or 2;
n is from 1 to 4 when m=1;
is from 1 to 6 when m=2;
x is from 0 to 3 when m=1, where n+x+z≦4,
is from 0 to 5 when m=2, where n+x+z≦6; and
z is from 0 to 3 when m=1, where n+x+z≦4,
is from 0 to 5 when m=2, where n+x+z≦6.

3. The rylenetetracarboximide of general formula I according to claim 1, in which the variables are each defined as follows:
R, R′ are each independently hydrogen;
C₁-C₃₀-alkyl whose carbon chain may be interrupted by one or more —O— and/or —CO— moieties and which may be mono- or polysubstituted by cyano, C₁-C₆-alkoxy, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic; C₅-C₈-cycloalkyl which may be mono- or polysubstituted by C₁-C₆-alkyl:
phenyl, naphthyl, pyridyl or pyrimidyl, each of which may be mono- or polysubstituted by C₁-C₁₈-alkyl, C₁-C₆-alkoxy, cyano, nitro, halogen, —CONR²R³, —SO₂NR²R³ and/or phenyl- or naphthylazo, each of which may be substituted by C₁-C₁₀-alkyl, C₁-C₆-alkoxy or cyano;
R¹ is hydrogen or C₁-C₆-alkyl;
R², R³ are each independently hydrogen;
C₁-C₁₈-alkyl which may be substituted by C₁-C₆-alkoxy, halogen, hydroxyl, carboxyl and/or cyano; aryl or heteraryl, each of which may be substituted by the above radicals specified for alkyl and by C₁-C₆-alkyl;
A is a 5- to 7-membered ring which is bonded via a nitrogen atom and whose carbon chain may be interrupted by one or more —O—, —S—, —NR¹—, —CO— and/or —SO₂— moieties, to which one or two unsaturated or saturated 4- to 8-membered rings may be fused, whose carbon chain may likewise be interrupted by these moieties and/or —N═, and the overall ring system may be mono- or polysubstituted by C₁-C₂₄-alkyl which may be substituted by aryl which may bear C₁ -C₁₈-alkyl as a substituent,
where the A radicals may be the same or different when m>1;
Z is phenoxy, phenylthio, pyridyloxy, pyrimidyloxy, pyridylthio or pyrimidylthio, each of which may be mono- or polysubstituted by C₁-C₁₂-alkyl which may be substituted by aryl, and/or aryl,
where the Z radicals may be the same or different when z>1;
m is 1 or 2;
n is from 2 to 4 when m=1;
is from 2 to 6 when m=2; and
z is from 0 to 2 when m=1, where n+z≦4,
is from 0 to 4 when m=2, where n+z≦6.

4. A process for preparing a rylenetetracarboximide of the general formula Ia 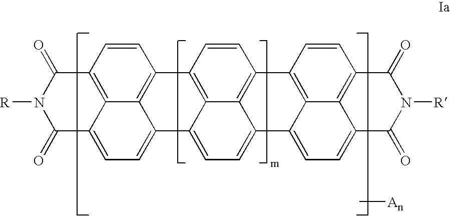
in which the variables are each as defined in claim 1, which comprises reacting a halogenated rylenetetracarboximide of formula IIa 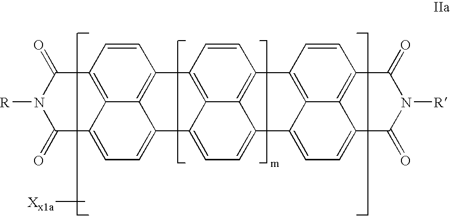
in which X and x1a are each defined as follows:
X is halogen; and
x1a is from 1 to 4 when m=1;
is from 1 to 6 when m=2,
optionally in the presence of a nonacidic solvent, with a cyclic amine of the formula III
H-A III
or a salt of this amine.

5. A process for preparing a rylenetetracarboximide of the general formula Ib 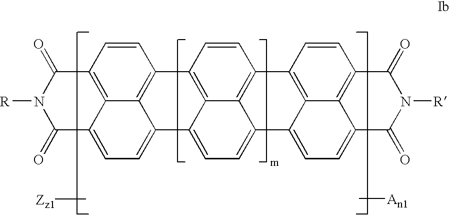
in which R, R′, A, Z and m are each as defined in claim 1 and n1 and z1 are each defined as follows:
n1 is from 1 to 3 when m=1;
is from 1 to 5 when m=2; and
z1 is from 1 to 3 when m=1 where n1+z1≦4;
is from 1 to 5 when m=2 where n1+z1≦6,
which comprises reacting a rylenimide of the general formula IIb 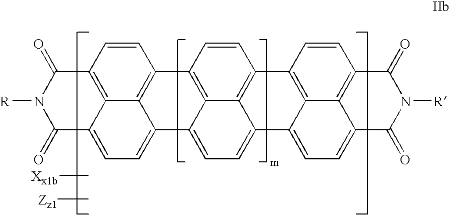
in which X and x1b are each defied as follows:
X is halogen; and
x1b is from 1 to 3 when m=1 where x1b+z1≦4;
is from 1 to 5 when m=2 where x1b+z1≦6,
optionally in the presence of a nonacidic solvent, with a cyclic amine of the formula III
H-A III
or a salt of this amine.

6. A colored high molecular weight organic or inorganic material comprising a rylenetetracarboximide of formula I according to claim 1.

7. The material according to claim 6, wherein the high molecular weight material is a coating material, printing ink or plastic.

8. An organic pigment comprising a rylenetetracarboximide of formula I according to claim 1 as a dispersion assistant and pigment additive.

9. An aqueous polymer dispersions which absorbs in the near infrared region of the electromagnetic spectrum comprising a rylenetetracarboximide of formula I according to claim 1.

10. A marking or inscription which absorbs infrared light and is invisible to the human eye comprising a material which comprises a rylenetetracarboximide of formula I according to claim 1.

11. An infrared absorber for heat management comprising a rylenetetracarboximide of formula I according to claim 1.
